(19) United States
(12) Patent Application Publication
(10) Pub. No.: US 2024/0315548 A1
(43) Pub. Date: Sep. 26, 2024

(54) COMPUTER PROGRAM, METHOD, AND APPARATUS FOR DETERMINING A VISUAL ACUITY OF A TEST PERSON

(71) Applicants: MACHINEMD AG, Bern (CH); UNIVERSITÄT BERN, Bern (CH)

(72) Inventors: Dominik BRÜGGER, Bern (CH); Dominic SENN, Bern (CH); Mathias ABEGG, Bern (CH)

(73) Assignees: MACHINEMD AG, Bern (CH); UNIVERSITÄT BERN, Bern (CH)

(21) Appl. No.: 18/579,516

(22) PCT Filed: Jul. 13, 2022
(86) PCT No.: PCT/EP2022/069623

§ 371 Date: Jan. 16, 2024

(30) Foreign Application Priority Data
Jul. 13, 2021 (EP) 21185449.2

(51) Int. Cl. A61B 3/032; A61B 3/00; A61B 3/08; A61B 3/11; A61B 3/113; A61B 3/18

(52) U.S. Cl. CPC A61B 3/032; A61B 3/005; A61B 3/0091; A61B 3/085; A61B 3/112; A61B 3/113; A61B 3/18

(57) ABSTRACT

The invention relates to a computer program causing a system with a computer to execute a method, particularly a computer-implemented method for determining at least one functional ocular parameter, namely a visual acuity, wherein the system comprises an optical system with a display system that is configured to independently project a visual stimulus to a left and a right eye of a test person, wherein the visual stimulus comprises a plurality of patches arranged on a background, an eye-tracking system configured to record a gaze direction of the left as well as the right eye, a computer configured to control the optical system and to receive recorded data from the eye-tracking system, the method comprising at least the steps of: executing a trial session for determining the visual acuity, wherein the trial session comprises a plurality of the visual stimuli that are presented, with the optical system to the left and/or the right eye of the test person; recording data during the trial session comprising information on the gaze direction; and determining from the recorded data of the trial session at least the visual acuity by analyzing a movement, particularly a correlation of a movement of the left and the right eye of the test person in response to the presented visual stimuli of the trial session, wherein the patches are characterized in that the patches are iso-luminant with respect to the background, that the patches exhibit a luminance distribution that periodically varies with a spatial frequency, and wherein each patch is a bounded set covering only a fraction of the background.

The invention relates to a computer program for executing a method, particularly a computer-implemented method for determining a visual acuity of a test person as well as a system configured to execute the computer program according to the invention.

In the art, methods for determining functional ocular parameters such as visual acuity, strabismus, a relative afferent pupillary defect, and a visual field are known.

Typically, the evaluation of these parameters requires various instruments and specialized medical personal, in order to perform the required tasks. This in turn prolongs the time required for estimating these parameters. Thus, the more parameters are to be determined from a test person, the higher the degree of compliance of the test person needs to be.

In addition, some methods, such as the swinging flashlight test for the determination of RAPD suffer from a high degree of variability of the test results upon repetition of the test.

Therefore, there is a need for methods that are less complicated in terms of test person compliance and understanding of the test and in terms of execution by the practitioner.

While virtual reality systems have addressed several issues related to user vision, see e.g. US 2017/0290504 A1, there is still a lack of computer-assisted diagnostic methods for functional ocular parameters.

With regard to visual acuity measurements, a problem in the art is the so-called Moiré-effect, that sets boundaries in terms of displayable items on a screen and that may interfere with a proper display of periodic patterns on a display.

The invention sets out to fill this gap. For this reason and in order to overcome the problems associated with the methods known in the art, the invention provides a computer program, a computer-implemented method and a system, particularly a neuro-ophthalmoscope that aim to resolve these issues.

Advantageous embodiments are described in the dependent claims.

According to the invention, a computer program that when executed on a computer, causes a system with the computer to execute a method, particularly a computer-implemented method for determining a visual acuity, wherein the system comprises an optical system with a display system that is configured to independently project a visual stimulus to a left and a right eye of a test person. Said display system may comprise a first and a second display system, such as a first and a second screen. The display system is configured to independently project a visual stimulus to a left and a right eye of a test person. The system further comprises an eye-tracking system configured to record a gaze direction and a pupil size of the left as well as the right eye simultaneously, independently and in a time-resolved fashion, particularly with a sampling rate, that is a frame rate of better than 20 Hz, particularly better than 50 Hz, more particularly better than 200 Hz. In addition, the system comprises a computer with a processor configured to control the optical system, for example to control an illumination brightness or a luminance and a duration of the visual stimuli on the display system, and to receive data from the eye-tracking system.

The method to be executed according to the invention comprises at least the steps of:
executing a trial session for determining the visual acuity of the test person, wherein said trial session comprises a plurality of visual stimuli that are presented, particularly simultaneously with the optical system to the left and/or the right eye of the test person;
recording data during said trial session for determining the visual acuity comprising information on the gaze direction;
determining from the recorded data of the trial session at least the visual acuity by analyzing a movement, particularly a correlation of a movement of the left and the right eye of the test person in response to the presented visual stimuli of the trial session for determining the visual acuity.

The visual stimulus comprises a plurality of patches arranged on a background, wherein the patches are characterized in that the patches are iso-luminant with respect to the background, and in that the patches exhibit a luminance distribution that periodically varies with a spatial frequency, particularly only one spatial frequency, and wherein each patch is a bounded set covering only a fraction of the background.

The trial session for determining the visual acuity may be configured such that initially there is no movement of the eyes and only once the test person recognizes the stimulus, movement of the left and the right eye sets in.

The invention may be claimed alternatively or additionally as a method, particularly a computer-implemented method. The computer-implemented method may be configured to interface with the components of the system in order to execute the method steps.

The invention allows for measuring visual acuity with a single trial session.

The optical system comprises a display system for displaying the visual stimuli to the test person's eye. For this purpose, the display system may comprise two display portions or two displays configured to present the visual stimuli to the left and/or the right eye. This presentation can be done independently for each eye, such that it is possible to present a visual stimulus to a selected eye only, while presenting no stimulus or a neutral stimulus to the other eye of the test person at the same time.

The display system may comprise two or more different computer-screens used for desktop applications. Alternatively, the display system may comprise only one computer-screen separated optically along a vertical line in two display screen sections.

The system may comprise a chin rest for the test person so that head movements relative to the eye-tracking system are reduced. Alternatively, the display system comprises one or more near-eye displays or a similar apparatus that is arranged directly in front of the eyes, e.g. at a distance shorter than 20 cm, with a virtual distance of between approximately 0.2 m and greater 4 m, particularly at infinite distance. Particularly, near eye-displays in form of head-up displays or virtual reality goggles (VR-goggles) may be worn by the test person during the execution of the method. This allows for a more precise and controlled test environment, particularly a chin rest may be omitted in such a system. In addition, the use of near-eye displays may allow to perform clinical tests with the system for which the test person is required to move the head while the gaze positions of the two eyes are recorded. Further, such a system can be designed as a portable system, which allows the use in field studies.

The system further comprises an eye-tracking system. The eye tracking system particularly has a temporal resolution in the millisecond range, which is equivalent to a frame rate in the range of several 100 Hz, such that a saccadic movement of the eye can be temporally resolved and recorded. Typically, the eye-tracking system has a frame rate or time resolution of 200 Hz or better.

However, for determining the visual acuity it is sufficient if the system has a lower temporal resolution e.g. in the range of 30 Hz to 60 Hz, As resolving the saccadic movements is not of particular importance.

The eye tracking system can be arranged at the display system or can be integrated in the near-eye display, e.g. in the VR-goggles or a similar apparatus.

The eye-tracking system is configured to record data on the size of the pupil and the gaze direction for each eye independently. The recorded data may be evaluated by the computer or by the eye-tracking system.

According to another embodiment of the invention, the trial session comprises plurality of trials.

In general, a trial session particularly comprises at least one sequence of trials, wherein a trial comprises at least one visual stimulus. Particularly, a trial involves the presentation of a visual stimulus to both eyes, either sequentially or simultaneously.

According to another embodiment of the invention, the patches are iso-chromatic.

This embodiment allows for providing colored stimuli that nonetheless allow for determining the visual acuity.

According to an alternative embodiment, at least some of the patches are of different color than the background.

According to this embodiment, the patches may not be iso-chromatic, but iso-luminant such that a test person may not be able to distinguish patches of different color in case the color vison is impaired.

The latter embodiment allows for testing a color vision of the test person simultaneously with or independently of the visual acuity.

According to another embodiment of the invention, the stimulus comprises a plurality of patches, wherein each patch is a bounded set that covers a limited area of the background of the patch. Particularly, a patch covers a connected area.

According to another embodiment of the invention, the patches are identical to each other, wherein the patches differ in a location at which they are displayed on the background. The patches according to this embodiment have the same size, shape and luminance distribution.

According to another embodiment of the invention, wherein the luminance distribution varies with a single spatial frequency.

This allows for a unique definition of the visual acuity of the test person, as compared to a plurality of spatial frequencies. The single spatial frequency allows for associating a single spatial frequency at which the test person is not perceiving the patches anymore.

Particularly, in case the periodically varying luminance distribution is generated by a supposition of a plurality of periodic functions, the periodic function comprising the largest amplitude is considered as the spatial frequency of the luminance distribution. The spatial frequency is considered to produce a luminance distribution with a periodically varying luminance. That is, for example, a Gaussian luminance distribution is not considered to be a periodic luminance distribution, despite the possibility to generate the Gaussian function with a superposition of a plurality of sinusoidal functions, which implicitly have a spatial frequency. The spatial frequency of the luminance distribution is considered the spatial frequency that provides the periodicity to the luminance distribution. Preferably the luminance distribution varies with a single spatial frequency and thus exhibits a unique periodicity. In certain embodiments, the spatial frequency is not smaller than a pixel of the display system.

According to another embodiment of the invention, the luminance distribution varies periodically at least along one direction with the spatial frequency. A direction may be chosen along a 2D plane of the display system and/or along a direction in an associated Cartesian Coordinate System in which the patches may be arranged in. Particularly, the periodically varying luminance distribution of each patch varies in a radial direction with respect to each patch. That is, the luminance of each patch May vary periodically and radially with respect to a center of the patch.

According to another embodiment of the invention, the spatial frequency is isotropic, such that the spatial frequency is the same along all directions of the patch during a trial of the trial session. This embodiment yields a radially symmetric periodically varying luminance distribution for each patch, such as concentric rings.

According to another embodiment of the invention, a spatially averaged luminance of the patch equals a luminance of a background of the stimulus on which the patches are displayed to the test person.

According to another embodiment of the invention, the visual stimulus is adjusted for different trials with respect to the spatial frequency of the luminance distribution.

Alternatively, or additionally, the visual stimulus is adjusted for different trials respect to a size of the patches.

Both embodiments in combination or alone allow to detect a threshold of a test person at which the test person may not be able to perceive the patches on the background anymore.

The two preceding embodiments define a trial session, wherein for each trial the spatial frequency and/or the size of the patches is adjusted, particularly wherein a direction of the spatial frequency of the luminance distribution remains the same.

According to another embodiment of the invention, the size of the patches and the spatial frequency of luminance distribution are adjusted for different trails such that a number of periods of the of the varying luminance distribution in the patch remains constant. This embodiment in essence discloses that the patches are scaled for different trials.

According to another embodiment of the invention, a ratio between the size and the number of periods comprised in the patches is constant for different trials.

The size may be an area covered by the patch, a diameter or an extent along the direction in which the spatial frequency of the patch varies.

According to another embodiment of the invention, between 0.5 and 100 periods of the periodically varying luminance distribution are comprised in the patch, particularly independent of the size of the patch. Preferably, each patch comprises one to five periods of the spatially varying luminance distribution. More particularly, each patch comprises more than one period.

The preceding embodiments allow to adjust the size of the patches while maintaining the same appearance of the patches, that is the patches are solely scaled up or down.

According to another embodiment of the invention, the patches are arranged irregularly and/or non-overlapping on the background.

According to another embodiment of the invention, during the trial session, the patches are moved back and forth at least once along a predefined trajectory, wherein the eye-tracking system records an eye movement and/or the gaze direction of the eye in response to the moving patches. Particularly, the trajectory may be a smooth trajectory, i.e. it does not comprised sudden, i.e. non-continuous speed changes neither in magnitude nor in direction.

The term “smooth” in the context of the current specification particularly relates to the property of the trajectory, profile or function to be differentiated in a mathematical sense. That is the trajectory, profile or function may not only be continuous but also differentiable.

The embodiment specifies the measurement process for determining the visual acuity.

According to another embodiment of the invention, the patches are moved back and forth along one direction, particularly a horizontal direction i.e. along a direction that extends parallel to an imaginary axis connecting the eyes of the test person.

According to another embodiment of the invention, the patches are moved back and forth following a periodic, particularly smooth periodic speed profile for each trial. The speed profile is the same for all patches. The speed profile may be circular, elliptic in terms of location, while e.g. having a constant magnitude of speed.

According to another embodiment of the invention, the speed profile may follow sinusoidal speed profile along one direction. That is, the patches move back and further along one direction while exhibiting a sinusoidal magnitude of speed.

According to another embodiment of the invention, during the trial session the patches are presented to one eye only and then subsequently to the other eye, such that a monocular acuity can be determined.

According to another embodiment of the invention, during the trial session, the size of the patches is decreased or increased successively for different trials, particularly after the patches have moved along the predefined trajectory at least once.

This embodiment allows to find the threshold at which a person starts or stops to perceive the moving patches.

This threshold may be determined with the following embodiment.

According to another embodiment of the invention, from a correlation of the recorded eye-movement to the movement of the patches, the visual acuity of the test person is determined, particularly wherein the visual acuity is determined from the patch size and/or the spatial frequency at which the eye movement deviates by a predefined value from the movement, particularly the trajectory of the patches, particularly wherein the deviation is measured in form of a least square deviation of the eye-movement relative to the patches movement. Alternatively, or additionally, a predefined correlation value, a lag time and/or a spatial lag of the eye position and the location of the patches may serve as the predefined value.

The eve-movement may follow the movement of the patches with a temporal offset. Therefore, said predefined value may be a maximum offset value, which when exceeded indicates that the person is no longer perceiving the patches reliably.

The visual stimulus may comprise a background, and the patches that are arranged on the background, wherein the stimulus may comprise noise. The predefined value may therefore depend on the noise.

Also, the predefined value may be given by a selected number of saccades that are performed by the eyes of the test person during a trial and that end on a position of the patch. If the number of saccades that do not end on a patch location exceeds a predefined number it may be concluded that the test person does not perceive the patches anymore.

According to another embodiment of the invention, the background consists of a uniform color or gray value having a uniform luminance.

According to another embodiment of the invention, the patches maintain a fixed distance and fixed relative position to each other, such that a pattern in which the patches are arranged with respect to each other on the background does not change when the patches are moving.

According to another embodiment of the invention, the patches are Gabor patches.

Gabor patches are particularly well-suited for determining the visual acuity.

According to another embodiment of the invention, the luminance of the Gabor patches follows a Gaussian kernel function having a variance, particularly an isotropic variance, wherein said Gaussian kernel function is modulated by a sinusoidal plane wave having a predefined direction and a predefined period.

This embodiment specifies the appearance of the Gabor patches further.

According to another embodiment of the invention, the period of the sinusoidal plane wave is in the range of the variance and the triple variance of the kernel function.

According to another embodiment of the invention, the sinusoidal plane wave is centered with its maximum amplitude or a zero-crossing at the mean value position of the Gaussian kernel function.

According to another embodiment of the invention, a ratio of the variance of the Gaussian kernel and the period of the sinusoidal plane wave of the Gabor patches is constant, particularly independent of the size of the Gabor patches.

According to another embodiment of the invention, the visual stimulus comprises a uniform background. The term ‘uniform’ particularly refers to a single color or gray background having a uniform luminance, such that the background does not exhibit any structural features.

According to another embodiment of the invention, the visual stimulus comprises, particularly consists of an area with a uniform color and luminance on which area the patches are randomly or irregularly arranged. Said area may be considered as the background of the stimulus. The background may remain unaltered throughout the trail session.

According to another embodiment of the invention, the luminance, particularly the luminance distribution, of the patch is given by a kernel function configured to define a size of the patch, wherein said kernel function is modulated by a periodic function configured to imprint the spatial frequency in the patch at which the luminance of the patch varies periodically. Particularly, the periodic function comprises exactly one spatial frequency.

The periodic function may be a sinusoidal function or a square wave function.

According to another embodiment of the invention, the period of the periodic function, particularly the period of the sinusoidal plane wave, is chosen such that the patch comprises between half a period up to three-periods within the size of the patch. Particularly, the period may be in the range of the variance to a third of the variance of the Gaussian kernel.

Particularly, the periodic function is centered with a maximum or minimum value at the patch.

For example, in case the patches are Gabor patches, the sinusoidal plane wave is centered with its maximum amplitude at the mean value position of the Gaussian kernel function.

According to another embodiment of the invention, a ratio of the variance of the Gaussian kernel and the period of the sinusoidal plane wave of the Gabor patches are constant, particularly independent of the size of the Gabor patches. This embodiment is a special case of a previous embodiment, that requires the ratio between size and number of periods in the patches to be constant. Thus, the variance of the Gaussian kernel function may be considered as the size or a measure of the size of the patch.

According to another embodiment of the invention, the patches, particularly the Gabor patches move along a direction, particularly along a horizontal direction or a vertical direction, wherein the speed of the patches changes periodically, particularly smooth periodically, more particularly sinusoidally, such that the patches move back and forth along said direction in a periodic fashion.

According to another embodiment of the invention, the periodic speed profile has a frequency between 0.02 Hz and 2 Hz, more particularly 0.05 Hz and 0.5 Hz.

According to another embodiment of the invention, the speed of the patches changes periodically, such that the patches move back and forth along said direction in a periodic fashion, such as for example zig-zag, or in a square-wave speed profile. The latter yields an abrupt change in in direction of the patches.

Particularly, the speed changes periodically with a smooth periodic function.

In the following, further embodiments that in combination with the invention may allow for a versatile and quick determination of visual ocular parameters other than the visual acuity.

According to a further embodiment of the invention the computer program is further configured to execute a method, particularly a computer-implemented method for determining a plurality of afferent and efferent functional ocular parameters, such as a relative afferent pupillary defect (RAPD), one or more strabismus angles, and/or a visual field with the system wherein an afferent pupillary defect (APD), particularly the relative afferent pupillary defect (RAPD) is determined from a first trial session consisting of a sequence of first kind of stimuli only that are presented in an automated fashion to the left and the right eye of the test person eyes with the display system, and/or wherein a visual field of the left and the right eye of the test person is determined from a second trial session consisting of a sequence of second kind of stimuli only, that are presented in an automated fashion to the left and the right eye of the test person with the display system, wherein from the first and/or the second trial session a strabismus is determined as well. Strabismus may be characterized by means of one or more strabismus angles, such as a horizontal strabismus angle, a vertical strabismus angle and/or a torsional strabismus angle. Therefore, this embodiment method allows to determine strabismus by determining one, two or all three of these strabismus angles. Particularly at least the horizontal strabismus angle is determined. In addition, it is possible to also determine the vertical strabismus angle. It is noted that any combination of the three strabismus angles may be determined when determining the strabismus of the test person.

This embodiment solves the problem to provide a shortened and simplified measurement method for various functional ocular parameters from a sequence of one kind of visual stimuli that allow for the determination of the plurality of functional ocular parameters using the same system. That is, during a single trial session, here the first or the second trial session a plurality of functional ocular parameters is determined in an objective fashion. Further, the method allows the measurement of the functional ocular parameters in an automated fashion without the need of the test person to provide outspoken or haptic feedback, which reduces complexity and eliminates error sources of the test. In addition, the fully automated execution may reduce measurement time for evaluating the different functional ocular parameters, as the test person and the medical personal does not need to switch between different measurement systems.

It is noted that in the context of the current specification a determination of APD may comprise the determination of RAPD as well, such that when the APD is determined at the same time or alternatively the RAPD may be determined from the same data set. For this reason, in the following, it is solely referred to APD instead of both variants.

Afferent and efferent ocular parameters, i.e. functional ocular parameters may comprise one or more of the group:
The APD and/or RAPD;
The strabismus angle;
A saccadic peak velocity;
An efferent pupillary function;
A saccadic accuracy;
A smooth pursuit;
A visual field particularly as determined by threshold perimetry;
A visual acuity;
A fusional amplitude.

The term “first” trial session as used herein is not to be understood as an indicator that the first trial session has to be the first trial session performed in a sequence of trial sessions, but solely serves the purpose of providing a distinction against a different trial session, such as the trial session for determining the visual acuity (which has no numbering associated with it), the second or the third session (which may be executed prior to or after the first trial session or the trial session for determining the visual acuity).

A visual stimulus is a stimulus that is presented to the eye of the test person. The stimulus may be visible to the test person or not—the latter happens for example, if the stimulus is a non-stimulus, dark stimulus or a neutral stimulus.

A visual stimulus of the first kind of stimuli may or may not comprise a fixation object or any object that allows focusing the eye on said object. Particularly, the first kind of stimulus comprises a predefined brightness, such that a pupil size would contract or dilate upon presentation of the first kind of stimulus.

Thus, the first kind of stimulus is designed to at least provide a predefined brightness stimulus, such that the eye to which the first kind of stimulus is presented, is exposed to a predefined and adjustable total brightness. By evaluating the contraction or the dilation of the pupil in response to the stimulus, several functional ocular parameters may be determined, such as the RAPD.

A first kind of stimulus that is expected to cause the pupil of the eye to contract is also referred to as a bright stimulus in the context of the specification, wherein a first kind of stimulus that is expected to cause the pupil of the eye to dilate is referred to as dark stimulus in the context of the specification.

Particularly, the dark stimulus/non-stimulus is represented by a black, particularly non-illuminated display state of the respective display system.

Particularly, the first kind of visual stimulus consists of a stimulus image.

Particularly, this first kind of visual stimulus differs from the stimuli presented during the trial session for determining the visual acuity.

During a central or middle part of the first trial session, the bright stimulus may be presented to one eye of the eyes, wherein simultaneously the dark stimulus may be presented to the respective other eye of the test person. Subsequently, the dark stimulus may be presented to the one eye simultaneous to the bright stimulus being presented to the other eye, wherein this procedure is repeated several times. The first kind of stimulus switches between the left and the right eye of the test person during the first trial session.

Particularly, in an initial and/or an end part of the first trial session, the bright stimulus may be presented to both eyes simultaneously followed by the dark stimulus presented to both eyes simultaneously. This initial part and/or the end part is particularly executed at a start of the first trial session before the central or middle part is executed and/or at an end of the first trial session after the central or middle part has been executed.

The execution of the initial and/or end parts in the first trial session may serve for the purpose of establishing and/or verifying baseline values for a constricted pupil size and a dilated pupil size for both eyes.

According to another embodiment of the invention, from the initial and/or the end parts of the first trial session, an efferent pupillary function or efferent pupillary defect is determined. Particularly, the efferent pupillary function is determined from a rate at which the pupils dilate after switching from the bright to the dark stimulus. In case the rate of dilation is below a predefined threshold rate, the test person might suffer from an efferent pupillary defect or an impaired efferent pupillary function, e.g. caused by Horner's disease.

During the first trial session the pupil size, for example the pupil diameter or the pupil radius, is automatically determined by the computer or the eye-tracking system from the data recorded by the eye-tracking system for each trial in a time-resolved fashion, such that for both eyes, a temporal course of the pupil size may be determined associated to the presented stimulus presented at any time of the temporal course.

By quantifying a change pupil size during the first trial session the ADP, and particularly the RAPD (as well as an efferent pupillary function) can be determined for each eye. Details of how APD may be determined from the measured data are elaborated at the example section.

Any trial session comprises trials that are based on similar kind of visual stimuli.

In particular, the first trial session consists of the first kind of stimuli only.

It is noted that the display position may be expressed in terms of a pair of angles describing the position on the display relative to a central gaze direction or a primary axis as is well known by the person skilled in the art.

According to another embodiment of the invention, executing the first trial session comprises the steps of:
Recording data with the eye-tracking system, wherein the data comprises information on a pupil size and a gaze direction of the first and the second eye of the test person during the first trial session, particularly wherein the sampling rate of the pupil size and gaze direction has a sampling rate of more than 100 Hz, particularly more than 200 Hz, and particularly wherein the data is recorded for at least one or more seconds;
Determining from the recorded data of the first trial session the APD by analyzing with the computer a temporal course of the pupil sizes of the left and the right eye, particularly in response to the presented first kind of stimuli;
Determining from the recorded gaze directions of the first trial session the at least one strabismus angle or more strabismus angles by analyzing with the computer a size and a direction of saccadic eye movements particularly in response to the presented first kind of visual stimuli.

According to another embodiment of the invention, executing the second trial session comprises the steps of:
Recording data with the eye-tracking system comprising information on the gaze direction of the first and the second eye of the test person during the second trial session, particularly wherein the sampling rate of gaze direction detection has a sampling rate of more than 200 Hz and particularly wherein the data is recorded for at least one or more seconds;
Determining from the recorded gaze directions of the second trial session the visual field and the at least one strabismus angle or more strabismus angles by analyzing with the computer saccadic eye movements of the test person particularly in response to the presented second kind of stimuli.

This embodiment allows for the determination of the visual field and simultaneously the determination of the strabismus of the test person by executing the second trial session having a different kind of visual stimuli as compared to the first kind of stimulus.

According to another embodiment of the invention, from the data recorded during the first and/or the second trial session and from the sequences of the first or the second kind of stimuli one or more of the following functional ocular parameters is or are determined from the data of the first and/or second trial session:
a saccadic peak velocity,
a saccadic accuracy.

This embodiment renders the method even more versatile, as the method using only one or two trial sessions (apart from the trial session for determining the visual acuity) with one or two kinds of visual stimuli allows the simultaneous determination of a plurality of functional ocular parameters using only a single measurement system.

According to another embodiment of the invention, from the recorded gaze directions of the first trial session the one or more strabismus angles is/are determined by analyzing with the computer a size and a direction of saccadic eye movements or the deviation of the gaze direction of the non-fixating eye from the gaze direction of the fixating eye.

Particularly, a deviation, for example a difference, of the gaze positions/directions of the two eyes is determined by the computer and from this deviation the strabismus particularly one or more strabismus angles are determined.

According to another embodiment of the invention, a deviation of the gaze positions/directions, particularly a difference between the gaze directions/positions of the two eyes is determined by the computer and from this deviation the strabismus particularly one or more strabismus angles are determined, wherein the deviation is determined at least once during a trial session, e.g. the first, second (third, or the trial session for determining the visual acuity) during trials in which one eye is exposed to a visual stimulus that is configured for fixating the eye on the stimulus and the other eye is not exposed to such a visual stimulus.

In the following, further advantageous embodiments are disclosed that allow the invention to measure even more functional ocular parameters on the same system using the same kind of stimuli.

According to another embodiment of the invention, an efferent pupillary function of both eyes is determined from the data of the sequence of the first kind of visual stimuli recorded during the first trial session.

According to another embodiment of the invention, a gain of the smooth pursuit is determined from data of the sequence of the second kind of visual stimuli recorded during the second trial session.

According to another embodiment of the invention, only the first trial session and the trial session for determining the visual acuity is executed.

This embodiment allows for a fast and reliable measurement session for determining two important functional ocular parameters, namely the APD and the strabismus angle of the test person by only executing a single trial session and the trial session for determining the visual acuity on the system.

According to another embodiment of the invention, exactly two trial sessions in addition to the trial session for determining the visual acuity are executed, namely the first and the second trial session, wherein at least the APD, the strabismus angle and the visual field of the test person are determined from the data of the sequences of the first and the second kind of visual stimuli recorded during the first and the second trial session. Particularly, the strabismus angle is determined either from the data recorded during the first or the second trial session only.

According to another embodiment of the invention, each visual stimulus of the first kind of visual stimuli comprises a fixation object that is displayed on the display system at a display location of the display system, wherein the fixation object is a spatially confined graphical object that allows fixation of an eye on the fixation object, particularly at one portion of the fixation object.

The spatially confined graphical object or a recognizable portion of a larger object is so small that the gaze direction when looking at the fixation object is indicative of the position of the fixation object within 0.1°. Particularly, the fixation object is smaller than 1°.

Particularly, the fixation object is an object that may be darker or brighter than a background of the stimulus image.

While for determination of the RAPD, the fixation object is of minor importance, the fixation object is necessary for the determination of the strabismus angle. Thus, when the strabismus angle is to be determined from the first trial session, the first kind of stimulus comprises the fixation object.

For example, by evaluating a saccadic amplitude and particularly a direction of the saccadic movement, when the first kind of stimulus comprising the fixation object is switched from the left to the right eye and/or vice versa, the strabismus angle can be determined and particularly quantifiably diagnosed by the computer from the measurement data.

The computer-generated measurement of the APD and the strabismus may be presented on an interface to a trained medical personal.

According to another embodiment of the invention, the first kind of visual stimuli are displayed alternatingly and repeatedly two or more times to the right and the left eye, particularly wherein the respective other eye is presented with the non-stimulus or dark stimulus, wherein the pupil size and the gaze direction of the test person are recorded for both eyes by the eye-tracking system, particularly at a frame rate of at least 100 Hz.

This allows the determination of the APD and the strabismus angle for each eye.

According to another embodiment of the invention, the first trial session comprises a plurality of gaze direction blocks, wherein in each gaze direction block the first kind of visual stimulus is repeatedly and alternatingly presented to right and the left eye, wherein the fixation object is displayed at different display locations for different gaze direction blocks, particularly, wherein for each gaze direction block the strabismus angle and the RAPD are determined separately.

The display location may be selected randomly from a set of display locations. However, it is possible to select display locations of the fixation object in response to a detected corrective saccadic eye movement of the eye, when the first kind of visual stimulus switched from one eye to the other eye.

Particularly, the display locations of the different gaze direction block are arranged over the visual field of the test person the display locations particularly in 3×3 matrix sectors, particularly wherein one of display locations, particularly a central display location corresponds to a primary gaze direction, i.e. a straight ahead gaze, wherein in each gaze direction block only one such sector is addressed by the display location. This way the strabismus angle may be measured during each gaze direction block for different sectors. The sectors may be referred to as combination of a vertical and horizontal general gaze direction, e.g. downward-right, upward only, left only, upward-left and so on. The primary gaze direction may be defined as the gaze direction that essentially is straight forward with respect to the head.

It is noted that during each of gaze direction blocks, data regarding to the pupil size for determining the APD is recorded.

As elaborated previously, during each gaze direction block, the first kind visual stimuli are presented repeatedly to the eyes, which according to this embodiment allows an averaging of the detected saccadic movements and pupils sizes, leading to more accurate results regarding the strabismus angle and the ADP from the first trial session.

In order to determine the strabismus in greater detail the following embodiment teaches an advantageous execution of the first trial session.

According to another embodiment of the invention, the fixation object is displayed at the same display location for the same gaze direction block, wherein an amplitude and a direction of the saccadic eye movement is determined from the recorded saccadic eye movement each time the first kind of stimulus switches from the left to the right eye or from the right to the left eye, particularly wherein the strabismus angle is determined for each gaze direction block, such that the strabismus angle is determined in relation to a gaze direction.

The detected saccadic eye movement may be evaluated by determining a saccadic starting point particularly for both eyes, that is recorded when the first kind of visual stimulus is switched from one eye to the other eye, and a saccadic end point that is determined for both eyes after the eye to which the fixation object is presented after the switch of stimulus had time to focus on the fixation object.

The direction defined by a vector pointing from the starting point to the end point provides information of the direction of the strabismus, wherein a length of said vector corresponds to the amplitude of the saccadic eye movement and may be associated to a magnitude of the strabismus.

In addition, it is possible to determine a torsional strabismus that relates to a torsion of the eyes. The torsion of the eye may be recorded by the eye-tracking system and evaluated by the computer from the data of the eye-tracking system, particularly also from a torsional state at a starting point of the saccadic eye movement and a torsional state at the end point of the saccadic eye movement. The torsion may be derived from torsional states at the starting and the end point of the saccadic movement.

The saccadic eye movement in response to the switch of the first kind of stimulus is particularly characterized by a direction pointing from the saccadic starting point to the saccadic end point, by an amplitude indicative for the distance between the saccadic starting point and the saccadic endpoint as well as by a torsion between the saccadic starting point and the saccadic end point, particularly wherein from at least one saccadic eye movement or from the plurality of the saccadic eye movements the strabismus angle is determined.

The strabismus angle may be expressed as a horizontal angle, a vertical angle and a torsional angle for each gaze direction.

It is noted that the term “same” display location with regard to a first and a second display system, refers to a corresponding display location on the first and the second display system that are deemed to correspond to identical gaze directions on an ideal eye model (no strabismus).

According to an alternative embodiment of the invention, wherein the size and the direction of the saccadic eye movement is determined from the recorded saccadic eye movement each time the first kind of stimulus switches from the left to the right eye or from the right to the left eye, wherein in a subsequent presentation of the first kind of visual stimulus to the same eye of the same gaze direction block, the display location of the fixation object is adjusted, such as to compensate the saccadic eye movement in amplitude and direction as determined from a previously presented first kind of stimulus of the gaze direction block, particularly until the saccadic movement is minimized in the same gaze direction block, particularly wherein the strabismus angle corresponds to the adjusted display location, particularly wherein the strabismus angle is determined for each gaze direction block, such that the strabismus angle is determined in relation to a gaze direction.

This embodiment allows for a more robust determination of the strabismus angle, as the method does not rely so much on accurate values from the eye-tracking but now only a non-movement of the eyes has to be established.

The terms and definitions of the previous embodiment apply also to this alternative embodiment.

This embodiment essentially aims to compensate a corrective saccadic eye movement by presenting the fixation object to the eyes in a shifted and/or rotated manner. This way, the strabismus magnitude, direction and particularly the torsion may be determined directly from the position and the degree of rotation of the fixation object.

The strabismus angle is particularly determined by measuring the amplitude and direction between the vector pointing from the starting point to the end point of the saccadic eye movement in response to the switch of the first kind of stimulus and assigning the size and the opposite direction to the adjusted display position to the strabismus angle.

According to this embodiment, a disparity of the visual stimuli presented to the two eyes is used to calculate the strabismus angle. Particularly, the disparity is defined as the direction and amplitude of the adjustments applied to of the fixation object to compensate the corrective saccadic eye movements. The strabismus angle may be expressed as a horizontal angle, a vertical angle and a torsional angle for each gaze direction.

According to another embodiment of the invention, each visual stimulus of the second kind of visual stimuli comprises a luminance object displayed on a uniform background at a relative position on the display system, wherein during the second trial session the luminance object of second kind of visual stimulus is displayed sequentially at a plurality of selected relative positions, wherein the second kind of visual stimuli are displayed alternatingly or sequentially to the right and the left eye, particularly wherein the respective other eye is presented with a neutral stimulus that is identical to the second kind of stimulus without the luminance object, wherein each time the luminance object is displayed at a selected relative position, it is determined by the computer, whether the test person has detected the luminance object at the selected relative position, wherein if the test person has detected the luminance object, the luminance object is subsequently displayed at a different selected relative position particularly with a decreased luminance, wherein the luminance object is displayed at the selected relative position again at a later trial with a decreased luminance (as compared to the luminance at which the test person detected the luminance object at the selected relative position), wherein if the test person has not detected the luminance object at the selected relative position, the luminance object is displayed repeatedly, but not necessarily subsequently, at the selected relative position with increasing luminance until the test person has detected the luminance object, such that for each selected relative position a luminance detection threshold for each eye of the test person is determined, such that the visual field is determined in form of a threshold perimetric measurement.

The relative position depends on a current gaze direction of the test person. The relative position is therefore is provided in a gaze-centric coordinate system. The current gaze direction may be determined by means of the eye-tracking system,

The relative position may be selected out of a plurality relative positions, particularly out of 20, 50 or more relative positions, that are distributed in quadrants of the visual field.

The luminance object is a spatially confined object that is so small that the gaze direction when looking at the luminance object is indicative of the position of the luminance object within 0.1°. Particularly, the luminance object is larger than 0.1°.

For example, the luminance object has a diameter size in the range of 0.1 to 1.7°, particularly a diameter size of 0.3° to 0.7°, and is arranged on a uniformly luminant background.

The luminance object may be adjusted in terms of its relative position and its luminance and/or size in the second kind of stimulus presented to the eye.

Particularly the luminance is decreased or increased between 2 dB to 10 dB, more particularly by 2, 4, 6, 8, or 10 dB, when the luminance is adjusted. The luminance of the luminance object may be controlled by the computer.

This measurement scheme with a luminance object that is positioned relative to the current gaze direction allows for a different kind of evaluation and test to be performed, where the test person does not need to permanently gaze in a specific direction. Therefore, this test setting requires less compliance of the test person with the test setup and the method execution. As will be discussed later, the determination of the visual field may be executed fully automated. Feedback, on whether the test person has detected the luminance object may be provided by the test person only be looking at the luminance object, as compared to executing an additional action, such as pressing a button, or speaking out loud. This renders the method more intuitive and thus less error prone.

The term luminance object particularly relates to the function of the object, namely to be able to change its luminance during the second trial session.

Therefore, the first kind of visual stimuli and the second kind of visual stimuli differ at least in that the luminance of the first kind of visual stimuli is not adjusted during the trial session.

The luminance object may be presented as a bright object on a uniform gray or black background with a continuous background luminance.

The second trials session allows for a threshold perimetry of the visual field. For this purpose, the relative positions may be selected randomly from the plurality of relative positions.

According to another embodiment of the invention, the luminance object is deemed detected by the test person, if a saccadic eye movement toward the displayed luminance object is recorded by the eye-tracking system within a first time interval during which the luminance object is displayed, particularly wherein the first time interval is around 500 ms, and wherein the luminance object stimulus is deemed not detected by the test person if no saccadic eye movement toward the luminance object is recorded by the eye-tracking system within the first time interval. The duration of the first time interval may be chosen such that it is at least so long that it comprises two standard deviations of average response times to visible stimuli, or particularly twice as long as the average response time.

Particularly, the second kind of visual stimulus, i.e. the luminance object is deemed detected by the test person only if a first saccade of the eye to which the luminance object is presented is bigger than 2°, more particularly bigger than 3° toward the relative position of the luminance object position.

More particularly, for each the second kind of stimulus an imaginary hit box is defined that extends around the position of the luminance object, wherein the size of the hit box depends on the position of the luminance object, wherein the size of the hit box is smaller for positions that are closer to a central region of the visual filed and bigger in a peripheral region of the visual field.

The determination of whether a visual stimulus has been detected, i.e. whether the luminance object was detected by the test person is performed automatically and reliably by determining whether there is a correlated saccade upon presentation of the luminance object and whether the saccade is going in the correct direction, i.e. toward the luminance object and also sufficiently along the correct direction toward the luminance object, so that incidental saccades may be excluded.

The saccadic movement of the eye toward the luminance object in case the eye detects the luminance object (i.e. if it is above the perception threshold of the eye at the relative position) allows for automatic determination of whether the test person has detected the luminance object upon display.

This embodiment allows for an automatic measuring of the visual field, without the test person having to provide a non-visual feedback, such as a spoken indication or pushing a button.

According to another embodiment of the invention, the strabismus angle is determined for the selected relative position from the saccadic movement and in particular from the amplitude and the direction of the saccadic eye movement, when the second kind of stimulus switches from the left to the right eye or from the right to the left eye, and if the luminance object is deemed detected by both eyes.

According to another embodiment of the invention, a gaze direction reset routine is performed, wherein said routine comprises the steps of:
particularly, determining whether the selected relative display position is located outside of a physical tracking limit of the eye tracking system or a physical display limit of the display system and if yes:
presenting a supra-threshold object, such as the fixation object or the luminance object with a supra-threshold luminance that lies above the luminance detection threshold at a current gaze direction of the eye,
moving the supra-threshold object to a new display location along a trajectory with a predefined horizontal and vertical speed,
particularly, hiding the supra-threshold object;
particularly, performing the method steps of the previous embodiment at the selected relative position that was determined to lie outside of the physical display limit or the physical tracking limit of the eye tracker before the gaze reset routine has been performed.

This gaze direction reset routine may be executed for example, if a selected relative position at which a trial is to be performed lies outside of the display limit, e.g. a physical lateral screen limit of the display system.

Further, from the gaze reset routine, a gain of smooth pursuit can be determined, by evaluating the movement of the eyes with respect to the moving supra-threshold object.

As such, the gaze reset routine may be executed during the first and/or the second trial session or any other trial session.

Also, the gaze reset routine may become necessary and useful, for example in case the visual field needs evaluation at a certain relative position, and most or all other relative positions for probing the visual field have been already tested. Then, the gaze direction of the test person is reset so that the relative position at which the trial is to be performed lies within the physical limits of the display system or within the physical tracking limit of the eye-tracking system.

According to another embodiment of the invention, during the gaze reset routine, the supra-threshold object with a predefined velocity pattern along a horizontal (from the test persons perspective) direction, and a vertically going direction particularly in a sequential fashion, wherein a deviation between a velocity of the detected eye movement following the supra-threshold object and the velocity pattern of the supra-threshold object is determined for each supra-threshold object movement direction, such that a smooth pursuit can be determined from the gaze reset routine.

According to this embodiment, for example in the second trial session the second kind of visual stimulus is selected with a luminance object having a luminance above a test person detection/perception threshold, wherein said luminance object is displayed to the test person at a current gaze direction, i.e. such that it is presented “directly” in front of the gaze direction, wherein said luminance object moves with the predefined velocity pattern. In this case the supra-threshold object corresponds to the luminance object.

Similarly, the routine may be performed and evaluated during the first trial session using the fixation object or during yet another trial session using a different object.

It is noted that the supra-threshold object may be a dark object on a bright background or vice versa.

This embodiment allows for example the integration of the determination of a smooth pursuit in the first or the second trial session by using the same first or second kind of visual stimulus.

According to another embodiment of the invention, the functional ocular parameters that are determined further comprise
a fusional amplitude of the test person;
   wherein for determining the fusional amplitude, the method further comprises the steps of:
Executing a third trial session, wherein the third trial session comprises a third kind of visual stimuli that are presented with the optical system simultaneously to the left and the right eye of the test person;
Recording data during the third trial session comprising information on the gaze direction;
Determining from the recorded gaze direction of the third trial session at least a fusional amplitude by analyzing with the computer a vergence of the left and the right eye of the test person in response to the presented third kind of visual stimuli of the third trial session.

According to another embodiment of the invention, the third kind of visual stimulus comprises an image having localized spatial structures (with a plurality of different spatial frequencies in each structure), wherein at the beginning of the third trial session the images for the left and the right eye are arranged at zero image disparity requiring zero vergence, wherein subsequently the images are presented to the left and the right eye in particularly continuously increasing or decreasing disparity, particularly wherein the disparity may be a horizontal disparity, a vertical disparity or a torsional disparity

This embodiment allows for the determination of the fusional amplitude by determining a maximum vergence angle of the eyes-horizontally and/or vertically—at which the test person may still compensate disparity of the images/displayed third kind of stimulus with a vergence eye movement. An increase of image disparity at this point breaks the fusion and vergence decays to its steady state, which ideally is at zero degree.

According to another embodiment of the invention, a gaze direction of the left and the right eye is recorded by the eye-tracking system during any of the trial sessions, wherein the strabismus angle is determined by subtracting the recorded gaze directions of the left and the right eye, particularly wherein the gaze direction of the eyes is recorded during a presentation of the first, the second, the third or the fourth kind of stimulus.

It is noted that either of the trial session may be performed independently from each other on the system.

According to a third aspect of the invention, a neuro-ophthalmoscope comprises the features of the system, as well as program code stored on the computer to execute the method according to the invention.

Further, the optical system and the eye-tracking system is comprised in a near-eye display, such as in VR-goggles or wherein the optical system comprises desktop screens and a separate eye-tracking system arranged at the computer screens, wherein in case the neuro-ophthalmoscope comprises the VR-goggles, said goggles further comprise for each eye a lens assembly that is adjustable such that optical aberrations of each eye of a test person may be compensated by the lens assembly.

Particularly, exemplary embodiments are described below in conjunction with the Figures. The Figures are appended to the claims and are accompanied by text explaining individual features of the shown embodiments and aspects of the present invention. Each individual feature shown in the Figures and/or mentioned in said text of the Figures may be incorporated (also in an isolated fashion) into a claim relating to the device according to the present invention.

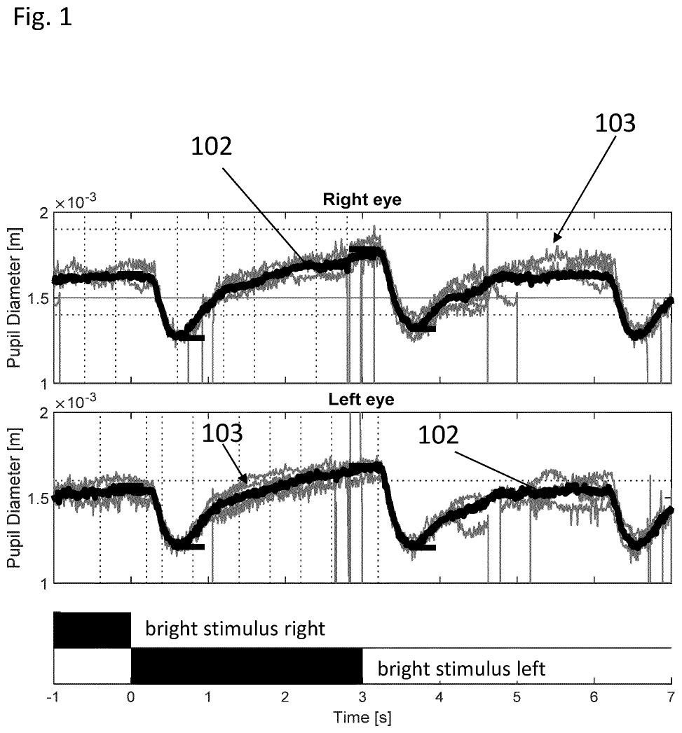

FIG. 1 shows a measurement of the pupil diameter according to the invention for determining RAPD;

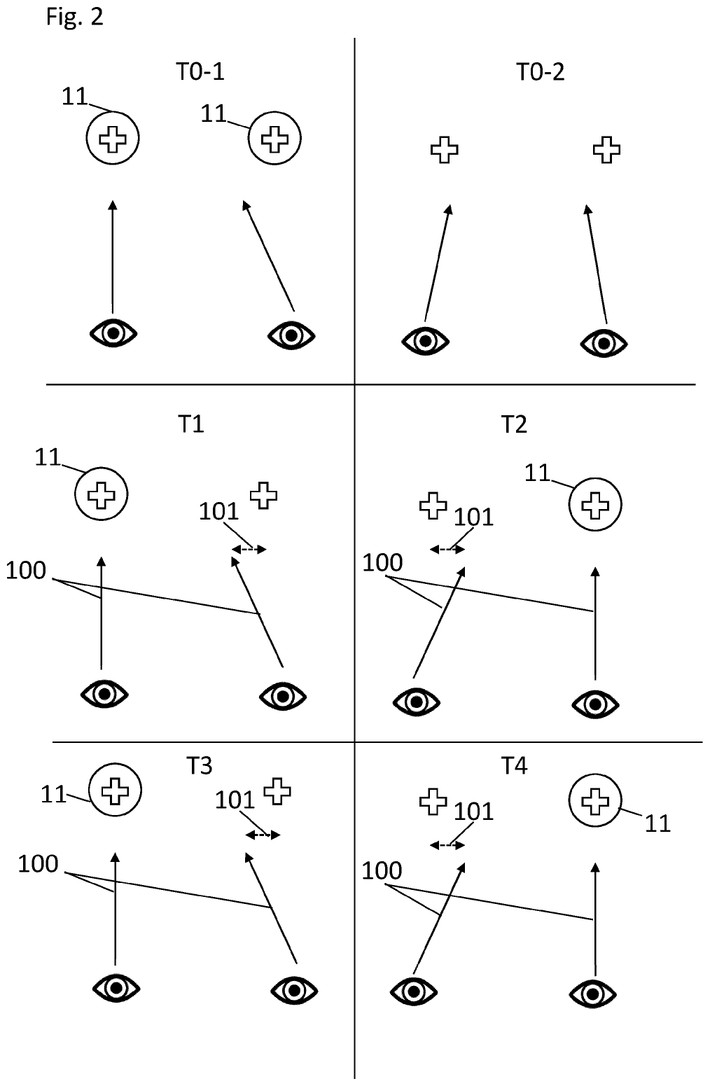

FIG. 2 schematically shows one embodiment of the invention for measuring strabismus;

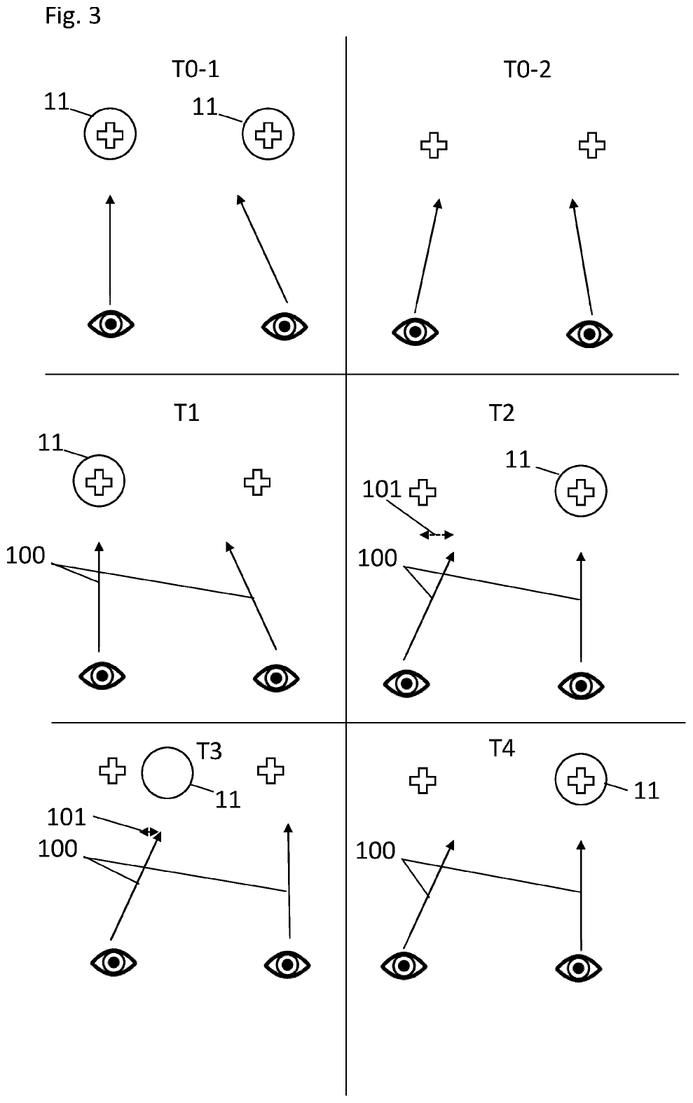

FIG. 3 schematically shows another embodiment of the invention for measuring strabismus;

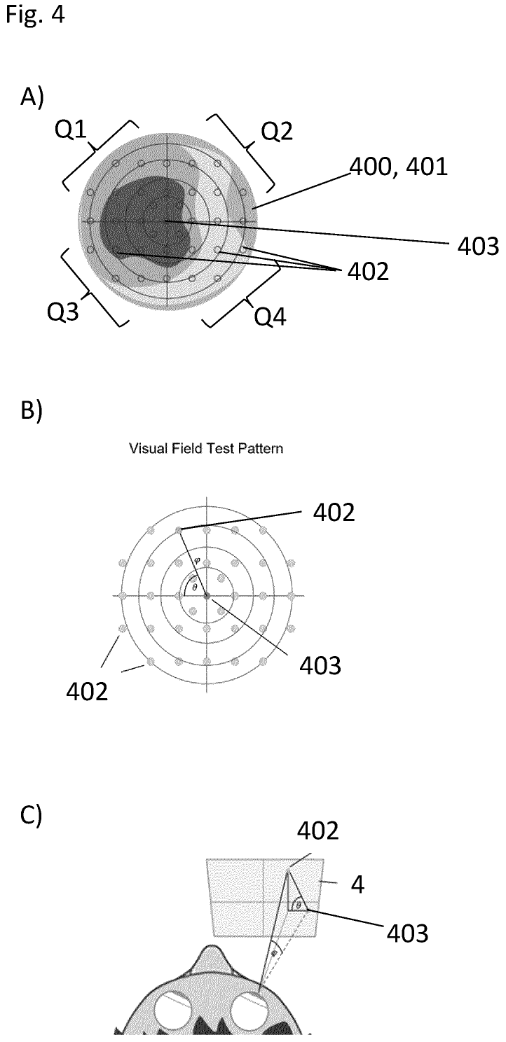

FIG. 4 schematically shows a measurement of the visual field of a person;

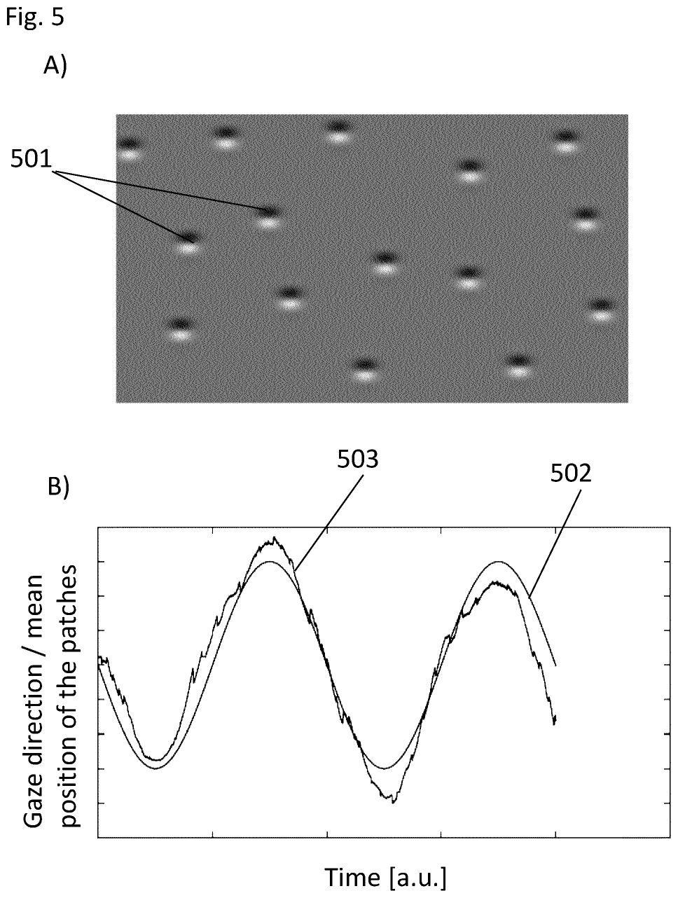

FIG. 5 shows the stimuli and the evaluation for determining the visual acuity of the person;

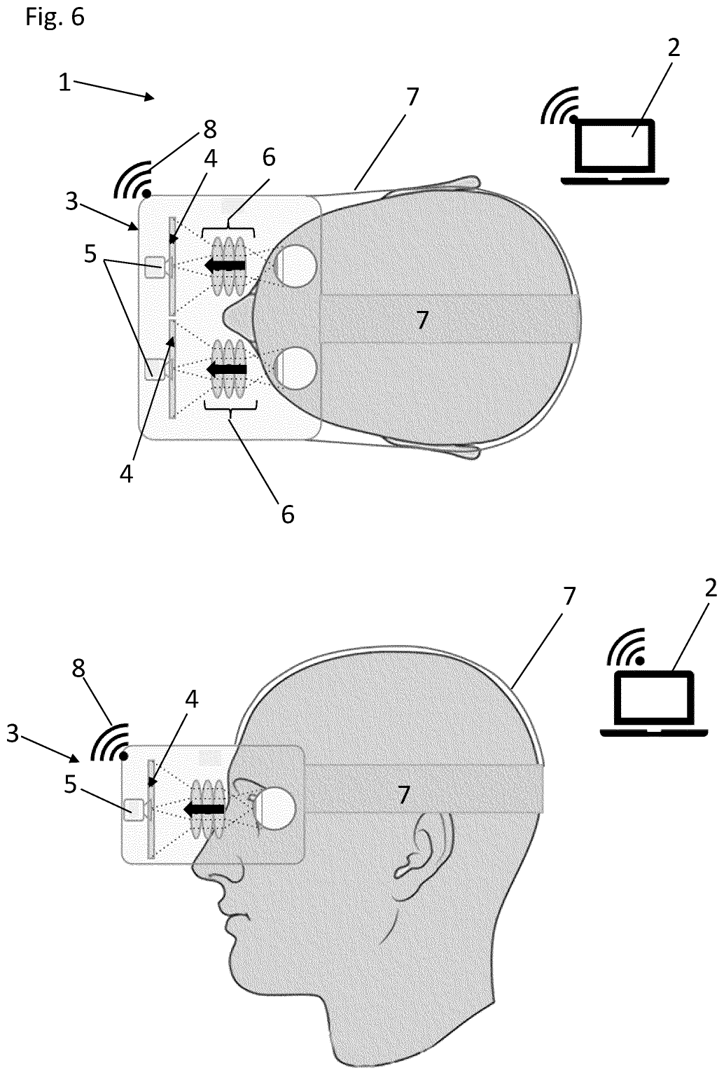

FIG. 6 schematically shows a system according to the invention; and

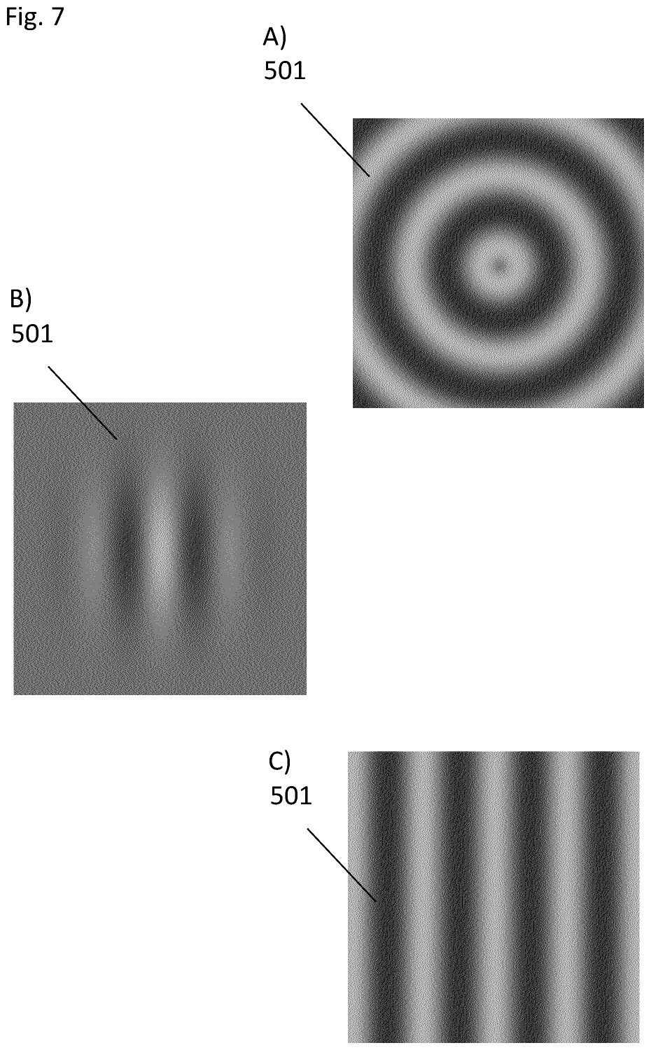

FIG. 7 various patches for the stimuli used for determining the visual acuity.

### EXAMPLES

In the following an exemplary system according to the invention is disclosed. However, it is noted that it is possible to use a different system that allows to execute the method according to the invention in the same fashion.

The system comprises a display system comprising two displays that are arranged in front of the eyes of the test person. The display system comprises two displays that are configured to independently display the visual stimuli to the eyes, wherein a first display of the displays is arranged to present the visual stimuli to the right eye and a second display of the displays is arranged to present the visual stimuli to the left eye.

Further, the displays are arranged such that the eyes may perceive only one display, such that the visual stimuli may be presented to one eye only. Any cross-talk between the displays should be below 0.2 cd/m².

The displays may have an associated coordinate system that accounts for a display position or display location, at which a visual stimulus or an object may be displayed.

The coordinate system is essentially the same for both displays and identical coordinates correspond to identical gaze directions with regard to an eye model having normal functional ocular parameters, i.e., a strabismus angle of zero degree.

Further, the system comprises an eye-tracking system that is configured to record a gaze direction of the eyes and a pupil size. In additional the eye-tracking system may be equipped to also determine a rotation of the eye and other relevant parameters.

The eye-tracking system has a frame rate higher than 200 Hz, such that saccades of the eyes may be resolved in a series of images.

The eye-tracking system may be integrated in the display system or be arranged in a vicinity to the display system. This ensures that the eyes of the test person are visible to the eye-tracking system at all times during the trial sessions.

The eye-tracking system may record data in form of images of the eyes of the test person. The eye-tracking system may comprise a module for determining several eye parameters, such as an eye position, a pupil position a pupil size and/or a gaze direction.

The module may transfer the eye parameters to the computer or include this data into the image data recorded from the eye-tracking system.

The precision of determining the pupil size should be better than 0.01 mm, while an accuracy may be better than 0.05 mm.

In addition, in order to determine a gaze direction sufficiently accurate the eye-tracking system should be configured to have an accuracy of better than 1°.

The eye-tracking system is connected to a computer of the system, such that the data may be exchanged between the computer and the eye tracking system.

The computer controls the eye-tracking system and the display system and is thus configured to execute the method according to the invention, particularly the computer-implemented method steps of the invention. The method may be executed on the computer in form of a computer program that causes the computer to control the display system and the eye-tracking system of the system such that the recording and displaying of visual stimuli is coordinated by the computer.

Further, the computer is configured to analyze the recorded data from the eye tracking system and in particular the computer may time-correlate the recorded data for the eye-tracking system with the presented visual stimuli.

The display system may comprise a lens assembly that is configured to be adjusted for myopia of the test person.

The display system and the eye tracking system may be comprised in a single device that is worn as a headset of the person, i.e. a near-eye display, for example in form of virtual reality goggles (VR-goggles). The VR-goggles may comprise the lens assembly.

The system according to the invention may then be used for executing the method in order to determine a plurality of functional ocular parameters.

According to the invention the trial session for determining the visual acuity may executed on the system for example by means of the computer program, a method or a computer-implemented method. The determination of the visual acuity is described in a later example.

Determination of APD, RAPD from the Pupil Size and the First Trial Session.

In the following for some functional ocular parameters, one or more examples are given that demonstrate in detail how the respective parameter may be determined.

Conventionally, the afferent pupillary defect (APD) is determined by the so-called swinging flashlight test that is prone to the expertise of the person executing and evaluating the test as well as to the compliance of the test person.

According to the invention, the test can be executed repeatedly and reliably in an automated and objective fashion.

The automated execution of the determination of the APD requires the system as described in the context of the specification.

For determining the APD, a first trial session is performed with the test person. The first trial session consists of a plurality of trials. A trial in turn consists of at least one first kind of visual stimulus that is presented to at least one eye of the test person. During the trial session the eye-tracking system continuously records the pupil size and the gaze direction of both eyes.

The visual stimulus used for the first trial session in this example is the first kind of stimulus with a fixation object, even though the fixation object is not needed for any specific purpose of the test with regard to the determination of the APD.

The first kind of stimulus is so bright, i.e. has such a high luminance, that a pupil of a healthy person, i.e. a person particularly not suffering from APD. is expected to constrict. The brightness is approximately 30 cd/m² but may be up to 50 cd/m².

Optionally, at the beginning or that the end of the first trial session, both eyes may be presented simultaneously with a non-stimulus or a dark stimulus that in essence allows the pupils of the test person to dilate for at least several seconds or minutes, T0-1, T0-2 (FIG. 2 and FIG. 3). From this measurement, a base line for the pupil size may be calculated from a temporal course of the recorded pupil sizes for each eye independently. Subsequently, the first kind of stimulus is presented simultaneously to both eye of the test person for several seconds such that the smallest pupil size of the constricted pupils for both eyes is determined from the temporal course of the recorded pupil sizes for both eyes independently.

After this optional initial sequence, for each eye the smallest and largest pupil size is known.

Then, a sequence of trials is presented to the test person, wherein during this sequence the first kind of stimulus, i.e. the bright stimulus is presented to the eyes in an alternating fashion, wherein the respective other eye is presented with the dark stimulus. That is for example starting with the left eye, the bright stimulus is presented to the left eye for example for a duration of three seconds and the dark stimulus is presented to the right eye for the same duration. Then, the bright stimulus is presented to the right eye and the dark stimulus is presented to the left eye for the same duration. The display location of the fixation object is only of secondary nature for the determination of the APD.

This sequence is repeated at least once. For the purpose of evaluating the recorded data, a frame rate of 60 Hz of the eye-tracking system may be sufficient.

From the first trial session, a temporal course of the recorded pupil sizes is obtained wherein said temporal course has several hundred to thousand data points.

From the temporal course, the maximal pupil size before the switch of the bright stimulus and the minimal pupil size just after the switch are determined by the processor, particularly wherein the temporal course for each eye is smoothed for outliers with a smoothing algorithm. Thus, from the temporal courses for both eyes, an associated amplitude of the change upon exposure to the first kind stimulus may be calculated by the processor. Thus, for each eye, the amplitude between constricted and dilated pupil is calculated. The difference of the pupillary response, i.e. the amplitudes between the left and the right eye indicates the magnitude of the relative afferent pupillary deficit (RAPD). The absolute amplitude compared to normal data from healthy subjects indicates the afferent pupillary deficit (APD). The latter is determined for each eye individually.

Optionally, the pupil size of the left eye and of the right eye are averaged. On the averaged traces a mean pupil size from 0 ms to 80 ms of an onset of the bright stimulus is determined. From this value the minimal pupillary size that is reached after the onset of the bright stimulus in the entire trial is subtracted. This calculation provides a pupillary amplitude. The pupillary amplitude of the left eye and the right eye are compared. The difference indicates the size of the RAPD.

A second approach consists of subtracting the mean pupillary size at the beginning and the end of each trial. This value is determined with a bright stimulus on the right eye is subtracted from the value with a bright stimulus on the left eye. The sign and magnitude of the resulting value indicates which eye is affected by RAPD and to what extent.

In FIG. 1, the temporal course according to the first trial session and the associated recorded temporal pupil sizes is shown. In this example a RAPD of the right eye is detected, as the amplitude of the change from the bright stimulus from the right eye to the left eye is larger than the amplitude of the pupil size from the change of the bright stimulus from the left eye to the right eye.

One of the advantages of the method is that within a single trial session using the same kind of visual stimulus also the strabismus angle may be determined.

### Determination of the Efferent Pupillary Function

At the beginning or the end of the first trial session, an initial part and an end part of the first rial session may be performed as elaborated for establishing a baseline for the constricted and the dilated pupil size. For this purpose, the bright stimulus may be presented to both eyes simultaneously followed by the dark stimulus presented to both eyes simultaneously or vice versa.

From the initial and/or the end parts of the first trial session, an efferent pupillary function or efferent pupillary defect is determined. Particularly, the efferent pupillary function is determined with the processor from the recorded temporal course of the pupil sizes of the eyes. The efferent pupillary function is determined from a rate or a time interval at which the pupils dilate after switching from the bright to the dark stimulus. In case the rate of dilation is below a predefined threshold rate or the time interval is longer than a predefined time interval, the test person might suffer from an efferent pupillary defect or an impaired efferent pupillary function, e.g. caused by Horner's disease.

The rate of dilation can be determined from a gradient of the temporal course or by determining the time interval that is required by the pupils to dilate after the stimulus has been switched from bright to dark.

Determination of a Strabismus Angle from the First Trial Session

Referring to FIGS. 2 and 3, for determining the strabismus angle from the first trial session in addition to the RAPD, the first kind of stimulus comprises the fixation object. The fixation object may be a spatially confined object that allows fixing the gaze to the fixation object. For example, the object has a diameter of object may be a bright spot or an object displayed in a scenery in the order of 1°.

During the first trial session, the first kind of stimulus is presented repeatedly and alternatingly to the left and the right eye as described on the preceding paragraphs in different trials T1, T2, T3, T4.

Eye- and pupil recordings from the eye-tracking system are filtered, such that blink-events are removed from the dataset. Data with eye movement velocities faster than 600°/s are removed from the dataset.

In a first embodiment of the determination of the strabismus angle, the following steps are performed:

The first trial session is subdivided in gaze direction blocks, wherein during each gaze direction block the fixation object is displayed at the same display location during the trials T1, T2, T3, T4, while in different gaze direction blocks the fixation object is displayed at different selected display locations. Typically, nine display locations are selected that from a 3×3 matrix centered at the primary gaze direction (i.e. straight forward at the center, 0,0). Each selected display location is a combination of one of three horizontal gaze directions selected from the tuple [−α, 0°, +α], while α is in the range of 10° to 25°, and one of three vertical gaze directions selected from the tuple [−β, 0°, +β], while β is in the range of 10° to 25°.

Thus, during the first trial session nine gaze direction blocks are executed during which the first kind of stimulus alternates between the right and the left eye as described for the APD determination, and while for each gaze direction block the fixation object is displayed at one of the selected display locations.

The recorded gaze direction 100 and pupil size data are evaluated with regard to APD as described before and with regard to the strabismus angle. For the strabismus angle, a corrective saccade 101 of the eyes is recorded each time the first kind of stimulus 11 switches from the left to the right eye and vice versa. The corrective saccade may be absent in case the test person does not suffer from strabismus. In case the test person suffers from strabismus the corrective saccade becomes prominent. The direction and the amplitude of the corrective saccade allows for quantifying the strabismus angle. Further, the strabismus angle may be determined in relation to its vertical, horizontal or torsional magnitude due to the selection of the display locations of the fixation object. Thus, a horizontal, a vertical and/or a torsional strabismus angle may be determined for the first trial session.

In particular, a horizontal gaze position/direction of the left eye is subtracted from a horizontal gaze position/direction of the right eye, when the gaze is directed towards one out of nine display locations. This yields in one value for a horizontal relative deviation, i.e. a horizontal strabismus angle in relation to the display position, i.e. the gaze direction for each of the nine display locations.

In the same fashion, a vertical strabismus angle and a torsional strabismus angle may be calculated for each of the nine display positions.

That is, the direction, the amplitude and the torsion of this corrective saccadic eye movement indicates the horizontal, vertical and torsional strabismus angle for each eye.

This embodiment allows for the determination of the APD and the strabismus angle using a single trial session with a single kind of visual stimulus, therefore, effectively merging two different tests into a single test series. This in turn reduces the time necessary to establish two functional ocular parameters.

In an alternative embodiment the strabismus angle is determined with the following steps:

The first trial session according to this alternative embodiment is subdivided in gaze direction blocks, wherein in different gaze direction blocks the fixation object is displayed at different selected display locations.

In a first gaze direction block, the first kind of object is displayed alternatingly and repeatedly to the left and the right eye as described in previous paragraphs. The stimulus comprises the fixation object at a display location.

However, a corrective saccade is evaluated after the switch of the first kind of visual stimulus from one eye, e.g. the left to the other eye, e.g. the right eye. Wherein according to the evaluated corrective saccade, the display location of the fixation object is adjusted for the next trial, when the visual stimulus is switched again from the one eye, e.g. the left eye to the other eye, e.g. the right eye, such that the display location compensates for the expected corrective saccade. During the first gaze direction block, it is the goal to adjust the display locations of the fixation object for the left and the right eye such that a corrective saccade is compensated in either switching direction. This adjustment of the display location of the fixation object is repeated for the subsequent gaze direction blocks in the same fashion.

Typically, nine display locations are selected from a 3×3 matrix centered at the primary gaze direction (i.e. straight forward at the center, 0,0). Each selected display location is a combination of one of three horizontal gaze directions selected from the tuple [−α, 0°, +α], while α is in the range of 10° to 25°, and one of three vertical gaze directions selected from the tuple [−β, θ°, +β], while β is in the range of 10° to 25°. According to the alternative embodiment, the display locations of the fixation object displayed to the right eye is adjusted according to the evaluated corrective saccade, as described above, while the display location of the fixation object displayed to the left eye is unchanged. Once the corrective saccade compensation is achieved for the right eye, the display location of for the left eye is adjusted, while the display location of the fixation object displayed to the right remains fixed.

The direction and the amplitude of the adjusted display location with respect to the unadjusted display location (i.e. the disparity) allows for quantifying the strabismus angle. Further, the strabismus angle may be determined in relation to its vertical, horizontal or torsional magnitude due to the selection of the adjusted display locations of the fixation object.

This embodiment allows for the determination of (R)APD and the strabismus angle using a single trial session with a single kind of visual stimulus, therefore, merging two different tests into a single test series. This in turn reduces the time necessary to establish two functional ocular parameters.

Both embodiments for determining the strabismus angle have in common that a total angle of phoria (i.e. strabismus angle) in each display location, i.e. gaze direction is determined. For each of the nine display locations a strabismus angle, particularly a horizontal and a vertical strabismus angle is calculated, wherein the strabismus angle is calculated for the situation, when the right eye fixates the fixation object a strabismus angle is determined for the left eye, and a vice versa, i.e. for the situation, when the left eye fixates the fixation object a strabismus angle is determined for the left eye.

Thus, during the first trial session nine gaze direction blocks are executed during which the first kind of stimulus alternates between the right and the left eye as described for the APD determination, and while for each gaze direction block the fixation object is displayed at one of the selected and particularly adjusted display locations, such that a strabismus angle is determined.

### Determination of the Visual Field.

Turning to FIG. 4, according to the invention, the method allows the determination of the visual field 400 (cf. FIG. 4A) of the test person simultaneously with the determination of the strabismus angle.

For this purpose, a second kind of visual stimulus is provided to the test person in a second trial session. It is noted that the second trial session may be executed before or after or instead of the first trial session.

The second kind of stimulus comprises a luminance object, that is a compact, i.e. spatially confined and localized object that may be displayed on the display system.

The second kind of stimulus consists of a uniform background on which the luminance object is displayed.

The second kind of stimulus is adjustable in terms of the luminance object and its luminance on the display system. Further, a relative position, also referred to as a relative display position in the context of the current specification, of the luminance object may be adjusted, such that the luminance object is displayed at a selected position in the visual field of the test person. Therefore, the location at which the luminance object is displayed on the display system depends on the gaze direction of the test person. The relative position is a position that is relative given relative to the current gaze direction of the eye. Therefore, the relative display position is also termed gaze-centric display, as the current gaze direction 403 determines the point of origin for the display position.

Thus, in order to display the luminance at a specific relative position, the relative position must fulfill two criteria in order be perceivable by the test person:
a) the display location for the relative position of the luminance object must be within the limits of the display system, otherwise it is not possible to display the luminance object.
b) the display location for the relative position of the luminance object must be within the limits of the eye-tracking system otherwise it is not possible to determine an eye position or a pupil size.

For evaluating the visual field 400 of the test person, a map of the visual field of the test person is generated that comprises spatially resolved information on a threshold value (cf. FIG. 4A in form of the gray values indicative of the perception threshold in the visual field map; the darker the lower the perception threshold), below which the test person does not perceive the luminance object if the luminance of the luminance object is below said threshold value at the relative position at which the test has been performed. This approach is also termed as threshold perimetry. Thus, the visual field of the test person is determined using a threshold perimetry method and results in a visual field map 401.

In order to generate said map 401, the threshold value is determined for a plurality of different relative positions 402 that are distributed over the visual field (cf. FIGS. 4A and 4B).

A trial for determining the visual field starts with the onset of the second kind of stimulus 403 having the luminance object displayed at a selected relative position of the plurality of relative positions and ends approximately 500 ms after an eye movement toward the luminance object is made. Such a trial is classified as “seen” by the test person or deemed detected. If no eye movement in the direction of the stimulus is made within approximately 1000 ms the trial is classified as “not-seen” by the test person or deemed undetected. In each trial one out of the plurality of relative positions is tested.

The luminance object consists for example of a bright dot of about 0.5° diameter shown on a uniform background of 10 cd/m2. The second kind of visual stimulus may be displayed for the duration of 200 ms.

The luminance of the luminance object is variable depending on the relative position of the luminance object and depending on whether or not the stimulus was deemed detected at its specific location in a preceding trial of the second trial session. Initially, an initial luminance of the luminance object is chosen 2 dB above an age-corrected normal value for that relative position. If the stimulus is deemed detected after the trial, the luminance of the luminance object is decreased by 2 dB in the a later but particularly not in the subsequent trial. Subsequently to a detected stimulus, the relative position of the luminance object is changed in the next trial. If the stimulus is not recognized, i.e. deemed not detected, the luminance of the luminance object is adaptively increased for example by 2 dB to 10 dB, particularly by 2, 4, 6, 8, or 10 dB. This procedure repeated until the threshold value is crossed once. Alternatively, the luminance of the luminance object may be adaptively increased or decreased, i.e. not by a fixed amount.

For determining the visual field, for example 54 relative positions of the eye-centric visual field may be tested selectively. As the luminance object is displayed at relative positions, display locations near the nasal step, along the vertical meridian and in central visual field are overrepresented. The relative positions cover a visual field of 120° in total diameter.

If it is not possible to display the luminance object at a selected relative position e.g. due to limitations of the display system or the eye-tracking system, another relative position of the eight relative positions is chosen. If only relative positions are left that have already been tested, a gaze reset procedure may be executed, during which a suprathreshold stimulus appears which has to be followed by subject.

For this purpose, a suprathreshold stimulus appears at the current gaze direction/position. This stimulus may be in form of the luminance object having a luminance above the threshold value and moves for example first horizontally at 8° per second to a new lateral position, followed by a vertical movement of 8° per second to a new vertical position.

Then, the luminance object (potentially having a lower luminance) is displayed at the relative position that was previously out of bounds.

For each relative position the perceptual threshold also referred to as the threshold value is determined. Perceived sensitivity is calculated for each relative position using the mean of the least bright stimulus deemed detected and the brightest stimulus deemed as not detected.

Determination of Smooth Pursuit from the Gaze Reset Procedure:

From the gaze reset procedure, a smooth pursuit may be determined as well, by evaluating movement of the eye in response to the moving supra-threshold object. For this purpose, recorded saccades of the eyes are removed from the smooth pursuit analysis. Right, left and vertical eye movements are separated. A mean difference between speed of eye-movement and speed of the supra-threshold object movement expressed in percent may be defined as gain. A gain of 100% means that the eye movement is perfectly following at supra-threshold object speed.

### Fixation, Gaze Holding, Square Wave Jerks, Nystagmus

Fixation may be determined during any trial session. For this analysis smooth pursuit eye movements during gaze reset and all saccades with an amplitude of >3° are excluded. In particular, saccadic responses after a newly presented stimulus are excluded.

Such fixation events are analyzed separately for five different relative positions in the eye-centric view, namely a right gaze, a left gaze, an up gaze and a down gaze as well as the primary gaze.

The fixation stability corresponds to a number of fixation changes of the eyes while the supra-threshold object is continuously present on the screen. This number is indicative for fixational losses per minute and the time spent within 3° of the supra-threshold stimulus (% on target).

In addition, or alternatively, a frequency of square wave jerks may be determined during the first and/or the second trial session. Square wave jerks are defined by two horizontal saccades of the same amplitude<5°±1°, an outgoing and a back-going saccade, separated by a time interval of 200 ms-400 ms. The number of such events per minute are counted independent of a presence of a fixation, a luminance or a supra-threshold object.

Presence of other (than square wave jerks) saccadic intrusions: All events with back-to-back saccades, i.e. two saccades without a fixation event in between are classified as saccadic intrusion. Similar to the square wave jerks, double saccadic pulses, horizontal ocular flutter, macrosaccadic oscillations, microsaccadic oscillations and opsoclonus is identified.

Presence of nystagmus: A drift of the eye position greater than 1° per second, with a constant direction lasting longer than 5 seconds is defined as nystagmus. The mean velocity of the slow phase is used as magnitude of nystagmus. This is determined in each out of five patient-centric gaze positions/directions, i.e. relative positions.

Gaze holding function: For this only the right gaze, the left gaze, the up gaze and the down gaze data is included. In analogy to nystagmus detection, it is determined whether an eye movement drift in the opposite direction of the gaze position/direction appears, such as a downward drift of the pupil during the up gaze, a right drift during left gaze etc. If a drift appears, gaze holding is considered abnormal. Drift is quantified with the same algorithm as nystagmus.

It is pointed out that all these parameters may be evaluated during a single trial session, which provides dramatically shortened measurement times.

### Determination of the Saccadic Accuracy

In addition, from any session, a saccadic accuracy may be determined from the sequence of the second kind of stimuli simultaneously to the strabismus angle and the visual field of the test person. For the determination of the saccadic accuracy only subsequent second kind of stimuli that are deemed detected and that are displayed at different relative positions are selected for analysis with the computer. This selection may also be done automatically by the computer.

The first saccade greater than 3° in the direction)(+20° of relative position at which the luminance object is displayed are used for analysis. The difference (in percent) between distance between the relative positions of the subsequent second stimuli and the size of the first saccade is calculated. This difference corresponds to the saccadic accuracy.

The saccadic accuracy is determined for right going saccades, left going saccades and vertical saccades separately.

For each of the three saccade types the median error (% of target amplitude) may calculated and a number (in percent) of hypermetric saccades. The latter are defined as saccades with an amplitude>10% of the target amplitude.

In a variation of the saccadic accuracy determination the above numbers are corrected for target amplitudes as large saccades are physiologically hypometric. The amplitude dependent mean age correlated hypometria is deducted from each trial.

### Determination of the Peak Velocity

For determining a peak velocity, particularly from the data recorded during the second trial session, saccadic eye movements are analyzed and a peak velocity of the eye movement during the saccade is determined. The peak velocity may be expressed in degree/see, even though the saccade is shorter than one second. In an additional, or an alternative embodiment, the saccades recorded during the first, third or fourth trial session are analyzed for the peak velocity in the same fashion.

### Determination of the Fusional Amplitude

For determining the fusional amplitude, a third trial session needs to be executed on the system, wherein the third trial session comprises a third kind of visual stimuli that are presented with the optical system simultaneously to the left and the right eye of the test person.

Thus, in addition to the first and the second trial session, a third kind of stimulus has to be presented to the test person.

The fusional amplitude is determined from the recorded gaze direction of both eyes during the third trial session.

The third kind of stimulus comprises an image or any structured object, that allows focusing.

Now this image is displayed to the test person in ever increasing disparity, particularly continuously increasing disparity, while the eye-tracking system records the gaze directions of both eyes. It is expected that the eyes perform a vergence movement in order to allow the disparity of the images to be compensated.

The disparity of the images is increased until the test person cannot perform a vergence movement of the eyes anymore and the gaze direction of the eyes in essence switch back to a straight or natural gaze direction.

The fusional amplitude corresponds to the maximum disparity of images presented to the test person at which the person is still capable to adjust the gaze directions of the eyes at the required vergence for compensating the disparity.

The fusional amplitude can be determined in a horizontal, a vertical, and/or along any other direction. The fusional amplitude may be given in terms of a maximum vergence angle between the eyes.

### Determination of Visual Acuity

For determining the visual acuity, the trial session that is illustrated in the following may be executed by means of an exemplary embodiment.

The visual acuity can be determined independently of any first, second and/or third trial session. The numbering of the trial sessions is provided solely as a means to distinguish the various trial sessions that may be executed according to the invention. By no means it is intended to imply an order of execution to these trial sessions.

Thus, the trial session for determining the visual acuity may be executed before or after any of the first, second or third trial session.

The trial session for determining the visual acuity comprises a plurality of visual stimuli that are presented during different trials with the optical system to the left and the right eye of the test person. Preferably, patches 501, particularly Gabor patches are presented to the left and right in a sequential manner such that for each eye, such that the visual acuity is determined separately for each eye.

The patches may be Gabor patches. In general, the patches should be iso-luminant, have the form of a bounded set and cover a connected area, and should comprise a periodic spatial luminance distribution, that may be associated with a spatial frequency imprinted on the patch. The luminance distribution is therefore completely comprised by the patch.

Each stimulus comprises a plurality of patches 501 that are displayed preferably in an irregular pattern on the display system to the test person on a uniform background. The patches are arranged preferably non-overlapping. On average the patches cover approximately 0.01% to 20% of the background, while α minimum distance should be twice the size or the diameter of the patch.

The patches may be displayed on a uniform, particularly a gray background, having a uniform luminance, wherein the patches 501 are displayed by means of brighter and darker regions on said background. However, the patches are required to essentially be iso-luminant with the background.

The term “iso-luminant” particularly refers to the property of the patch that when the luminance of the patch is spatially averaged over the patch, said averaged luminance equals the background.

The term “iso-chromatic” particularly refers to the property of the patch that when the color of the patch is spatially averaged over the patch, said averaged color equals the color of the background.

The plurality of patches is presented simultaneously in each of the stimuli. In each trial, the patches 501 have identical properties except the position on the display system. The terms “position” and “location” in the context of the patches refer to the same property.

A patch may be characterized by its size, shape and luminance distribution. In particular, the Gabor patches are defined by a Gaussian kernel function having a variance, particularly an isotropic variance, such that the Gabor patches appear as circular objects, wherein said Gaussian kernel function is modulated by a sinusoidal plane wave having a predefined direction and a predefined period/spatial frequency. The size of the Gabor patch may therefore be the variance of the Gaussian Kernel, wherein the shape is circular, as the variance is isotropic, and the periodicity of the luminance distribution is provided by the periodic function, namely the sinusoidal plane wave function.

The period of the periodic function may be considered a wavelength of the periodic function.

For example, the frequency of the sinusoidal plane wave of such a Gabor is in the range of the single variance and the triple variance. But other frequencies may be chosen. Advantageously, the spatial frequency and the variance may be chosen such that one period is comprised by the Gabor patch, i.e. the variance and the period are for example identical but at least proportional to each other. The sinusoidal plane wave may be centered with its maximum amplitude at the mean value position of the Gaussian kernel function or alternatively with its zero-crossing. The ratio of the variance of the Gaussian kernel and the period of the sinusoidal plane wave of the Gabor patches may be constant. This allows for the display of Gabor patches of different sizes during different trials, while the appearance of the Gabor patches remain essentially the same, but scaled bigger or smaller.

Alternatively, instead of Gabor patches one may use patches comprising bright and dark concentric rings forming a ring pattern (cf. FIG. 7A), wherein an outer ring of each patch defines the size of the patch (by the means of the diameter of the outer ring) and the number of bright and dark rings may define the spatial frequency of the patch or more precisely the periodic function. The kernel function may be a circular step function. The rings are iso-luminant.

For different trials the diameter together with the “width” of the rings and thus the frequency of the ring pattern may be adjusted such that neither the number of rings nor the luminance does change.

Relating again to the properties of the patches in general, the patches comprise a periodic pattern or shape that is given by a periodic function, wherein said periodic function is modulated onto a kernel function that is configured to provide the size as well as the outer contour and an unmodulated brightness or luminance distribution.

The periodic function may be a sinusoidal wave function but may be chosen as a square wave function or triangular wave function. It is noted that the periodic function may be associated to only one spatial frequency, that is even a square wave function may be mathematically expressed by means of a superposition of sinusoidal functions, the square wave function exhibits a period that is unique and which is referred to when referring to a period of the spatial frequency.

For determining the visual acuity, the patches, particularly the Gabor patches of an initial size are moved back and forth on the display system along a predefined trajectory 502, wherein the eye-tracking system records an eye movement 503 of the eye to which the patches are displayed. The trajectory may comprise a movement back and forth along one axis or direction. The speed profile of said movement may be sinusoidal or another periodic but continuous or smooth speed profile.

For example, the size of the patches may be chosen so small that initially the test person does not detect the patches and thus no eye-movements are recorded. Any occurring saccades may be evaluated with regard to an end position, wherein if the saccades end at a position where a patch is displayed the patch might be counted as detected or perceived by the test person.

In case the test person does not detect the moving patches, the patch size is increased while the frequency may be lowered inversely, such that the ratio of patch size and number periods per patch remains constant.

Alternatively, or additionally, the trial session may be started with a patch size that is so big that the test person detects the patches and follows the movement of the patches, in subsequent trials the patches become successively smaller and the spatial frequency higher until the eyes stop following the movement of the patches or until a lag or correlation between movement of the patches and the eyes exceeds a predefined value.

The predefined trajectory may be a linear, i.e. along one direction, motion with a sinusoidal velocity profile 502 that is the same for all displayed patches. For example, the trajectory can comprise two to three periods of periodic motion until a new trial is executed, e.g. with smaller or bigger patches. The maximum speed during a periodic motion may not exceed 20°/sec, particularly may not exceed 10°/sec.

It is expected that the eye follows said trajectory as long as or as soon as the patches 501 are perceived by the test person.

In an evaluation step, the movement of the eye may be filtered for any saccadic movement of the eye, and only movement of the eye 503 that are not saccadic are analyzed for the visual acuity. The filtered eye movement and the motion of the patches are compared in terms of a correlation and a lag. In case the test person perceives the patches, both motions should be almost identical, while in case the test person does not perceive the patches, e.g. due to a too small patch size, the eye movement does not correlate sufficiently or lags significantly with respect to the motion of the patches. In FIG. 5B the test person is capable to follow the patches as can be seen when comparing the movement 502 of the Gabor patches in relation to the eye movement 503.

But as disclosed in a previous paragraph, the saccadic movements may alternatively or additionally be analyzed with respect to their end and/or starting point. In case the end and/or starting points coincide with a location of a patch, the patch may be considered to be perceived by the test person.

While typically, the visual acuity is estimated by means of an optotype (for example the “Landolt C”), this embodiment allows for an alternative determination of the visual acuity by means of moving patches. As the patch is in essence a small patch of stripes, it is also subject to the Moiree effect or interference. But since particularly the Gabor patch is comparably small, interference effects on the display are also small. In contrast, if one would choose stripes that cover the entire screen, the displayed stripes might appear much larger than intended, as the intended stripes displayed by means of the discrete pixels of a screen could cause such an effect. This will not happen with the patches, particularly the Gabor patches according to the invention.

In FIG. 6 a schematic view of one exemplary embodiment the system 1 according to the invention is shown. The system 1 comprises an external computer 2 that is connected with an optical system 3 comprising a display system 4, an eye-tracking system 5 a lens assembly 6, a fixation mechanism 7 for attaching the optical system 3 to the head 8 of a test person. In an alternative embodiment, the optical system may be comprised in a device that is arranged and configured to be arranged in front of the eyes of the test person, such that fixation mechanism is necessary.

Further, the optical system comprises a transmitter device 8 for wireless transmission of recorded data to the external computer. The transmitter device 8 is also configured to receive instructions from the external computer 2 so that the external computer 2 can control the optical system 3. The optical system 3 may comprise a non-transitory memory storage (not shown) for storing computer program instructions and recorded data. Further, the optical system 3 may further comprise a processor (not shown) configured to execute the computer program stored on the memory storage. The processor is further configured to be controlled by the external computer 2, i.e. to receive instructions from the external computer 2, to send confirmations and to control the display system 4 as well as the eye-tracking system 5 of the optical system 3. This allows for a fully automated execution of any trial session of the method according to the invention.

### The Optical System:

The optical system 3 comprises a housing that allows for a forming a light-tight enclosed volume between the eyes of the test person and the display system 4 and that houses the components of the optical system 3.

Further, the optical system 3 comprises a lens assembly 6 for adjusting the optical power and for compensating optical aberrations of the test person. The lens assembly 6 is adjustable in terms of its optical power and in terms of a virtual distance correction of the displayed stimuli or images. For this purpose, the lens assembly 6 may comprise spherical and cylindrical lenses. The eye-tracking system 5 may consist of two cameras, wherein each camera is configured and arranged to record one eye of the test person. The cameras are for example infrared cameras. The display system 4 comprises two displays or display portions-one for each eye.

The data recorded by the eye-tracking system 5 is stored in the memory of the optical system 3 such that it can be temporally associated with the stimuli presented of the eyes of the person.

### The External Computer:

The external computer 2 may be a conventional computer configured to receive user input and to display data on a display of the computer 2. The computer 2 comprises a transmitter device as well for transmitting and receiving information in a wireless fashion. Via the transmitter device the external computer 2 may be connected to the optical system 3.

FIG. 7A) to C) shows various exemplary embodiments of the patches. FIG. 7A) shows a circular patch that comprises three concentric rings of high luminance, which are spaced equidistantly apart from each other, such that a radial spatial frequency is imprinted on the patch.

FIG. 7 B) shows a Gabor patch, wherein the periodic function is sinusoidal plane wave along a single direction of the patch, wherein from the plane wave three extreme values are visible within the Gaussian kernel. The maximum intensity of the plane wave is at the center of the Gaussian kernel.

The patch fades into the background which is of uniform gray and has no “hard” boundary.

FIG. 7 C) is a square-shaped patch with a sinusoidal plane wave along one direction of the square, such that the intensity of the patch is modulated along said direction. The patch has a clearly recognizable boundary.

1. A computer program comprising a non-transitory computer-readable storage medium having program code embodied therewith, the program code executable on at least one hardware processor, causing a system with a computer to execute a method, particularly a computer-implemented method for determining at least one functional ocular parameter, namely a visual acuity, wherein the system comprises an optical system with a display system that is configured to independently project a visual stimulus to a left and a right eye of a test person, wherein the visual stimulus comprises a plurality of patches arranged on a background, an eye-tracking system configured to record a gaze direction of the left as well as the right eye, a computer configured to control the optical system and to receive recorded data from the eye-tracking system, the method comprising at least the steps of:
executing a trial session for determining the visual acuity, wherein the trial session comprises a plurality of the visual stimuli that are presented, with the optical system to the left and/or the right eye of the test person;
recording data during the trial session comprising information on the gaze direction;
determining from the recorded data of the trial session at least the visual acuity by analyzing a movement of the left and the right eye of the test person in response to the presented visual stimuli of the trial session,
wherein the patches are characterized in that the patches are iso-luminant with respect to the background, that the patches exhibit a luminance distribution that varies periodically with a spatial frequency, and wherein each patch is a bounded set covering only a fraction of the background.

2. The computer program according to claim 1, wherein the patches are identical to each other, wherein the patches differ in a location at which they are displayed on the background.

3. The computer program according to claim 1, wherein the patches have the same size, shape and luminance distribution.

4. (canceled)

5. The computer program according to claim 1, wherein the luminance distribution varies at least along one direction with the spatial frequency.

6. The computer program according claim 1, wherein the spatial frequency is isotropic, such that the spatial frequency is the same along all directions of the patch during a trial of the trial session.

7. The computer program according to claim 1, wherein a spatially averaged luminance of the patch equals a luminance of the background.

8. The computer program according to claim 1, wherein at least some patches are iso-chromatic with the background.

9. The computer program according to claim 1, wherein at least some patches the patches are of different color than the background.

10. The computer program according to claim 1, wherein the visual stimulus is adjusted for different trials at least with respect to the spatial frequency of the luminance distribution of the patches.

11. The computer program according to claim 1, wherein the visual stimulus is adjusted for different trials at least with respect to a size of the patches.

12. The computer program according to claim 9, wherein the size of the patches and a spatial frequency of luminance distribution are adjusted for different trials such that a number of periods of the varying luminance distribution in the patch remains constant.

13. The computer program according to claim 9, wherein a ratio of the size and the period of the spatial frequency of the patches is constant.

14. (canceled)

15. The computer program according to claim 1, wherein the patches are arranged irregularly over and/or non-overlapping on the background.

16. The computer program according to claim 1, wherein during the trial session, the patches are moved back and forth at least once along a predefined trajectory, wherein the eye-tracking system records an eye movement and/or the gaze direction of the eye in response to the moving patches.

17. The computer program according to claim 16, wherein the patches are moved back and forth following a periodic speed profile for each trial.

18. (canceled)

19. The computer program according to claim 1, wherein from a correlation of the recorded eye-movement to the movement of the patches and/or from a correlation of the recorded eye-positions with the locations of the patches, the visual acuity of the test person is determined, wherein the visual acuity is determined from the patch size or the spatial frequency of the patch at which the eye movement deviates by a predefined value from the movement or the position of the patches.

20. (canceled)

21. The computer program according to claim 1, wherein the patches maintain a fixed distance and fixed relative position to each other, such that a pattern in which the patches are arranged with respect to each other on the background does not change when the patches are moving.

22. The computer program according to claim 1, wherein the patches are Gabor patches.

23. The computer program according to claim 1822, wherein the luminance of the Gabor patches follows a Gaussian kernel function having a variance, wherein said Gaussian kernel function is modulated by a sinusoidal plane wave having a predefined direction and a predefined period.

24. (canceled)

25. (canceled)

26. The computer program according to claim 19, wherein a ratio of the variance of the Gaussian kernel and the period of the sinusoidal plane wave of the Gabor patches is constant.

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. A neuro-ophthalmoscope system, comprising:
an optical system comprising a display system that is configured to independently project a visual stimulus to a left and a right eye of a test person,
an eye-tracking system configured to record a gaze direction and a pupil size of the left as well as the right eye, a computer configured to control the optical system and to receive recorded data from the eye-tracking system, as well as program code stored on the computer to execute the computer program according to one the preceding claims,
wherein the optical system and the eye-tracking system comprises in a near-eye display, such as in VR-goggles,
wherein the near-eye display further comprises for each eye a lens assembly that is adjustable such that optical aberrations of each eye of a test person may be compensated by the lens assembly.
